SEQUENCE LISTING (1 sequence)

SEQ ID NO: 1 - protein, 28 aa, Homo sapiens
NVHSGSTFFKYYLQGAKIPKPEASFSPR

(19) United States
(12) Patent Application Publication
(10) Pub. No.: US 2005/0095611 A1
(43) Pub. Date: May 5, 2005

(54) Identification of biomarkers for detecting pancreatic cancer

(76) Inventors: Daniel W. Chan, Clarksville, MD (US); Zhen Zhang, Dayton, MD (US); Jens Koopmann, Bochum (DE); Michael Goggins, Baltimore, MD (US); C. Nicole White, Baltimore, MD (US); Eric Fung, Mountain View, CA (US); Xiao-Ying Meng, Fremont, CA (US)

Correspondence Address: Peter F. Corless;EDWARDS & ANGELL, LLP, P.O. Box 55874, Boston, MA, 02205, US

(21) Appl. No.: 10/836,649

(22) Filed: Apr. 30, 2004

Related U.S. Application Data
(60) Provisional application No. 60/467,501, filed on May 2, 2003.
(60) Provisional application No. 60/542,618, filed on Feb. 5, 2004.

(51) Int. Cl. C12Q 1/68; G01N 33/574

(52) U.S. Cl. 435/6; 435/7.23

(57) ABSTRACT

The present invention relates to a method of qualifying pancreatic cancer status in a subject comprising: (a) measuring at least one of the disclosed biomarkers in a sample from the subject and (b) correlating the measurement with pancreatic cancer status. The invention further relates to kits for qualifying pancreatic cancer status in a subject.

This application claims the benefit of U.S. provisional application No. 60/467,501, filed May 2, 2003, and U.S. provisional application No. 60/542,618, filed Feb. 5, 2004, both of which applications are incorporated herein by reference in their entirety.

### FIELD OF THE INVENTION

The invention provides biomarkers important in the detection of pancreatic cancer and for the reliable detection and identification of biomarkers, important for the diagnosis and prognosis of pancreatic cancer. The serum protein profile in pancreatic cancer patients are distinguished from non-neoplastic individuals using SELDI analysis. This technique provides a simple yet sensitive approach to diagnose pancreatic cancer using serum or plasma samples.

### BACKGROUND OF THE INVENTION

Pancreatic adenocarcinoma currently has the lowest survival rate for any solid cancer (1, 2). Despite progress in the understanding of etiology and pathogenesis of pancreatic adenocarcinoma, the 5-year survival of patients with pancreatic cancer has increased only marginally from 1% to 3-5% overall in the last decade (1, 3). Patients with surgically resectable cancers have the best hope for cure as they can achieve 5-year survival of 15-40% after pancreaticoduodenectomy (4). Unfortunately, only 10-15% of patients present with small, resectable cancers. Despite improvements in diagnostic imaging, diagnosis may be delayed in some patients for a variety of reasons including the presence of a small cancer (1, 3), the presence of a cancer that diffusely infiltrates the pancreas without forming a mass, because of delayed access to diagnostic services such as endoscopic ultrasound and fine needle aspiration, or because of the low sensitivity of cytology from fine needle aspiration. An accurate serological test could facilitate the rapid diagnosis of pancreatic cancer. Such a test would also be helpful for individuals with an increased risk of pancreatic adenocarcinoma, such as families with familial pancreatic cancer due to germline mutations in BRCA2, p16, those with hereditary pancreatitis and Peutz-Jeghers syndrome. There is no effective screening test for these individuals and the lifetime risk of developing pancreatic cancer in some of these at-risk groups can range from 10-70% (5-7). Unfortunately, the most widely used serum marker for pancreatic cancer, CA 19-9, is not sufficiently accurate to be useful as a diagnostic test, especially for identifying patients with small surgically resectable cancers.(8, 9). Its main utility is in monitoring the effects of treatment in patients known to have pancreatic cancer.

Recent advances in mass spectrometry are accelerating the identification of protein markers of disease and these advances have led to a new field of discovery, proteomics, often defined as the complete characterization of proteins in a biological sample (10, 11). One mass spectrometry platform for proteomic analysis is SELDI (surface-enhanced laser desorption and ionization) which resolves proteins in biological samples by placing samples onto biochemically distinct Protein Chips (Ciphergen Biosystems, Inc., Fremont, Calif.) and subjecting them to time-of-flight mass spectrometry. The SELDI technique requires that an energy absorbing matrix be applied to a biological sample on the protein chip so that when laser energy is applied to the sample, the proteins in that sample become ionized enabling their mass to be measured from the speed at which they travel through a positively charged vacuum. By using ProteinChip surfaces with different biochemical characteristics and by first fractionating proteins in a biological sample one can achieve a sensitive, high-throughput analysis of proteins in complex biological specimens such as serum. SELDI profiling has been successfully used to differentiate ovarian, breast and prostate cancer from controls (12-16), as well as to identify markers of bladder cancer in urine (17), and to identify a marker of pancreatic cancer in pancreatic juice (18). In addition to SELDI, the availability of effective bioinformatics tools to extract the maximum information usable for biomarker discovery has been key to identifying novel proteins (19-21).

A need therefore, exists which can specifically identify pancreatic cancer, can distinguish pancreatic cancer from pancreatitis, and identify the stages of disease progression.

### SUMMARY OF THE INVENTION

The present invention provides, for the first time, novel protein markers that are differentially present in the samples of human cancer patients and in the samples of control subjects. The present invention also provides sensitive and quick methods and kits that can be used as an aid for diagnosis of human cancer by detecting these novel markers. The measurement of these markers, alone or in combination, in patient samples provides information that diagnostician can correlate with a probable diagnosis of human cancer or a negative diagnosis (e.g., normal or disease-free). All the markers are characterized by molecular weight. The markers can be resolved from other proteins in a sample by using a variety of fractionation techniques, e.g., chromatographic separation coupled with mass spectrometry, or by traditional immunoassays.

In preferred embodiments, the method of resolution involves Surface-Enhanced Laser Desorption/Ionization (“SELDI”) mass spectrometry, in which the surface of the mass spectrometry probe comprises adsorbents that bind the markers.

In other preferred embodiments, comparative protein profiles are generated using the ProteinChip Biomarker System from patients diagnosed with pancreatic cancer and from patients without known neoplastic diseases. A subset of biomarkers was selected based on collaborative results from supervised analytical methods. Preferred analytical methods include the Classification And Regression Tree (CART), implemented in Biomarker Pattern Software V4.0 (BPS) (Ciphergen, CA), and the Unified Maximum Separability Analysis (UMSA) procedure, implemented in ProPeak (3Z Informatics, SC).

In a preferred embodiment, the analytical methods are used individually and in cross-comparison to screen for peaks that are most contributory towards the discrimination between non cancer diseases of the pancreas; organ confined pancreatic cancer; non-organ confined pancreatic cancer; pre-invasive stages of pancreatic cancer; malignant versus benign forms of cancer; different cancer stages of pancreatic cancer; and the non-cancer controls.

In another aspect, the biomarkers were purified and identified. The selected biomarkers, are evaluated individually and in combination through multivariate logistic regression. The biomarkers are also evaluated together with known tumor markers such as, for example, CA 19-9. Known markers such as CA 19-9 can be measured by any number of methods such as SELDI or immunoassay.

While the absolute identity of these markers is not yet known, such knowledge is not necessary to measure them in a patient sample, because they are sufficiently characterized by, e.g., mass and by affinity characteristics. It is noted that molecular weight and binding properties are characteristic properties of these markers and not limitations on means of detection or isolation. Furthermore, using the methods described herein or other methods known in the art, the absolute identity of the markers can be determined.

The present invention also relates to biomarkers designated as Markers I through XXXII. Protein markers of the invention can be characterized in one or more of several respects. In particular, in one aspect, these markers are characterized by molecular weights under the conditions specified herein, particularly as determined by mass spectral analysis. In another aspect, the markers can be characterized by features of the markers' mass spectral signature such as size (including area) and/or shape of the markers' spectral peaks, features including proximity, size and shape of neighboring peaks, etc. In yet another aspect, the markers can be characterized by affinity binding characteristics, particularly ability to binding to an IMAC nickel adsorbent under specified conditions, however, other metals, e.g., copper, may also be used. In preferred embodiments, markers of the invention may be characterized by each of such aspects, i.e. molecular weight, mass spectral signature and IMAC3-Ni²⁺ absorbent binding.

For the mass values of the markers disclosed herein, the mass accuracy of the spectral instrument is considered to be about within +/−0. 15 percent of the disclosed molecular weight value. Additionally, to such recognized accuracy variations of the instrument, the spectral mass determination can vary within resolution limits of from about 400 to 1000 m/dm, where m is mass and dm is the mass spectral peak width at 0.5 peak height. Those mass accuracy and resolution variances associated with the mass spectral instrument and operation thereof are reflected in the use of the term “about” in the disclosure of the mass of each of Markers I through XXXII. It is also intended that such mass accuracy and resolution variances and thus meaning of the term “about” with respect to the mass of each of the markers disclosed herein is inclusive of variants of the markers as may exist due to sex, genotype and/or ethnicity of the subject and the particular cancer or origin or stage thereof.

Molecular weights as measured by mass spectrometry are specified for each marker as follows:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD.

In one embodiment, the molecular weight for Marker II was previously determined to be 7.451 kD. In another embodiment the molecular weight for Marker III was previously determined to be 3.144 kD. These values fall within the mass accuracy range of the spectral instrument, as discussed above.

Markers I-XXIII also are characterized by their mass spectral signature. The mass spectra of each of Markers I-XXIII are set forth in FIGS. 2 through 20.

Each of Markers I-XXXII also is characterized by its ability to bind to an ProteinChip adsorbent (e.g., either IMAC-Cu⁺⁺ or WCX), as specified herein.

More specifically, Marker IV (molecular weight of about 12.8 kD) was discovered and subsequently identified, in accordance with the methods described herein, as full-length serum amyloid A protein (referred to herein as “SAA”). Marker III was discovered and subsequently identified, in accordance with the methods described herein, as a fragment derived from inter-a-trypsin inhibitor heavy chain H4 (referred to herein as “ITIH4”). The peptide sequence of Marker III was determined to be NVHSGSTFFKYYLQGAKIPKPEASFSPR (SEQ ID NO:1).

Preferred methods for detection and diagnosis of cancer comprise detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects, wherein the one or more protein markers are selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, or combinations thereof.

In a preferred embodiment, the present invention provides for a method for detecting, diagnosing and differentiating between pre-invasive, benign, malignant or different malignant stages of cancer, wherein the method comprises using a biochip array for detecting at least one biomarker in a subject sample; evaluating at least one biomarker in a subject sample, and correlating the detection of one or more protein biomarkers with cancer.

Additionally, as further discussed below, the invention provides methods for qualifying pancreatic cancer status in a subject that comprise measuring a biomarker selected a member of a protein cluster comprising:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker )a: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and combinations thereof, and

correlating the measurement with pancreatic cancer status. In certain preferred embodiments, the biomarker is selected from a modified protein cluster of Markers I through XXII, which includes all modified forms of the specified markers, but exclude the specific protein itself.

Preferably, one. or more protein biomarkers are used for detecting, diagnosing and differentiating between pancreatic cancer and other non-malignant pancreatic diseases, particularly one or more of the following biomarkers:
Marker I: having a molecular weight of about 3.667 kD
Marker II: having a molecular weight of about 7.441 kD
Marker IV: having a molecular weight of about 12.861 kD
Marker IX: having a molecular weight of about 3.473 kD
Marker X: having a molecular weight of about 5.903 kD
Marker XI: having a molecular weight of about 8.563 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD

Preferably, one or more protein biomarkers are used to determine whether the pancreatic cancer is at a pre-invasive stage or to identify the different malignant stages of pancreatic cancer. Also preferred is a detection of a plurality of the biomarkers, wherein at least about two biomarkers are detected.

A preferred group of biomarkers for use in accordance with the invention, employed either alone or in combination:
Marker XXIV: having a molecular weight of about 3.370 kD
Marker XXV: having a molecular weight of about 3.441 kD
Marker XXVI: having a molecular weight of about 10.055 kD

The accuracy of a diagnostic test is characterized by a Receiver Operating Characteristic curve (“ROC curve”). An ROC is a plot of the true positive rate against the false positive rate for the different possible cutpoints of a diagnostic test. An ROC curve shows the relationship between sensitivity and specificity. That is, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test, while an area of 0.5 represents a less useful test. Thus, preferred biomarkers and diagnostic methods of the present invention have an AUC greater than 0.50, more preferred tests have an AUC greater than 0.60, more preferred tests have an AUC greater than 0.70.

Preferably, the biomarkers of the invention are detected in samples of blood, blood plasma, serum, urine, tissue, cells, organs and seminal fluids.

Preferred detection methods include use of a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In preferred methods of the present invention, the step of correlating the measurement of the biomarkers with pancreatic cancer status is performed by a software classification algorithm. Preferably, data is generated on immobilized subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in pancreatic cancer patients and are lacking in non-cancer subject controls.

Preferably the biochip surfaces are, for example, ionic, anionic, comprised of immobilized nickel ions, comprised of a mixture of positive and negative ions, comprised of one or more antibodies, single or double stranded nucleic acids, proteins, peptides or fragments thereof, amino acid probes, or phage display libraries.

In other preferred methods one or more of the markers are measured using laser desorption/ionization mass spectrometry, comprising providing a probe adapted for use with a mass spectrometer comprising an adsorbent attached thereto, and contacting the subject sample with the adsorbent, and; desorbing and ionizing the marker or markers from the probe and detecting the deionized/ionized markers with the mass spectrometer.

Preferably, the laser desorption/ionization mass spectrometry comprises: providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be, hydrophobic, hydrophilic, ionic or metal chelate adsorbent, such as, nickel or an antibody, single- or double stranded oligonucleotide, amino acid, protein, peptide or fragments thereof.

In another embodiment, a process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers. Fractionation is monitored for purity on normal phase and immobilized nickel arrays. Generating data on immobilized marker fractions on an array, is accomplished by subjecting said array to laser ionization and detecting intensity of signal for mass/charge ratio; and, transforming the data into computer readable form; and executing an algorithm that classifies the data according to user input parameters, for detecting signals that represent markers present in cancer patients and are lacking in non-cancer subject controls. Preferably fractions are subjected to gel electrophoresis and correlated with data generated by mass spectrometry. In one aspect, gel bands representative of potential markers are excised and subjected to enzymatic treatment and are applied to biochip arrays for peptide mapping.

In another aspect one or more biomarkers are selected from gel bands representing:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD.

Purified proteins for screening and aiding in the diagnosis of pancreatic cancer and/or generation of antibodies for further diagnostic assays are provided for. Purified proteins are selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD.

The invention further provides for kits for aiding the diagnosis of cancer, comprising:

an adsorbent attached to a substrate, wherein the adsorbent retains one or more biomarker selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD.

Preferably, the kit comprises written instructions for use of the kit for detection of cancer and the instructions provide for contacting a test sample with the absorbent and detecting one or more biomarkers retained by the adsorbent.

The kit provides for a substrate which allows for adsorption of said adsorbent. Preferably, the substrate can be hydrophobic, hydrophilic, charged, polar, metal ions.

The kit also provides for an adsorbent wherein the adsorbent is an antibody, single or double stranded oligonucleotide, amino acid, protein, peptide or fragments thereof.

Detection of one or more protein biomarkers using the kit, is by mass spectrometry or immunoassays such as an ELISA.

In another preferred embodiment biomarkers, purified on a biochip and identified by their molecular weights, are selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD.

In another preferred embodiment, at least two purified biomarkers comprise a composition of a combination of any of the Markers I through XXXII for use in differentiating between diseases of the pancreatic, pancreatic cancer, and the different stages of pancreatic cancer.

Preferably each of the markers in the compositions are purified.

In further embodiments, the invention provides methods for identifying compounds (e.g., antibodies, nucleic acid molecules (e.g., DNA, RNA), small molecules, peptides, and/or peptidomimetics) capable of treating pancreatic cancer comprising contacting at least one biomarker selected from the group consisting of
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and
   combinations thereof with a test compound; and determining whether the test compound binds to the biomarker, wherein a compound that binds to the biomarker is identifies as a compound capable of treated pancreatic cancer.

In another embodiment, the invention provides methods of treating pancreatic cancer comprising administering to a subject suffering from or at risk of developing pancreatic cancer a therapeutically effective amount of a compound (e.g., an antibody, nucleic acid molecule (e.g., DNA, RNA), small molecule, peptide, and/or peptidomimetic) capable of modulating the expression or activity of at least one biomarker selected from the group consisting of
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and combinations thereof.

Other aspects of the invention are described infra.

### BRIEF DESCRIPTION OF THE FIGURES

FIG. 1a: An example of a 3-D plot of cancer (red) and normal control (green) subgroup separation in the component analysis module of ProPeak (IMAC, fraction 1).

FIG. 1b: An example of peak ranking in the BootStrap analysis module of ProPeak. Ranking represents the relative contribution of each m/z peak (bar) to the separation of the data (IMAC, fraction1).

FIG. 2 shows the mass spectra of Marker I from WCX F1. The mass spectral peak of Marker I is designated within the spectra with a vertical line.

FIG. 3 shows the mass spectra of Marker II from WCX F1. The mass spectral peak of Marker II is designated within the spectra with a vertical line.

FIG. 4 shows the mass spectra of Marker III from WCX F1. The mass spectral peak of Marker III is designated within the spectra with a vertical line.

FIG. 5 shows the mass spectra of Marker IV from WCX F1. The mass spectral peak of Marker IV is designated within the spectra with a vertical line.

FIG. 6 shows the mass spectra of Marker V from WCX F1. The mass spectral peak of Marker V is designated within the spectra with a vertical line.

FIG. 7 shows the mass spectra of Marker VI from WCX F1. The mass spectral peak of Marker VI is designated within the spectra with a vertical line.

FIG. 8 shows the mass spectra of Marker VII from WCX F1 and Marker VIII from WCX F1. The mass spectral peaks of Markers VII and VIII are designated within the spectra with a vertical line at the specified molecular weight, i.e. the peak of Marker VII is shown at the left of the spectra and the peak of Marker VIII is shown at the right of the spectra.

FIG. 9 shows the mass spectra of Marker IX from WCX F6. The mass spectral peak of Marker IX is designated within the spectra with a vertical line.

FIG. 10 shows the mass spectra of Marker X from WCX F6. The mass spectral peak of Marker X is designated within the spectra with a vertical line.

FIG. 11 shows the mass spectra of Marker XI from WCX F6. The mass spectral peak of Marker XI is designated within the spectra with a vertical line at the left of the spectra.

FIG. 12 shows the mass spectra of Marker XII from WCX F6. The mass spectral peak of Marker XII is designated within the spectra with a vertical line.

FIG. 13 shows the mass spectra of Marker XIII from WCX F6 and Marker XIV from WCX F6. The mass spectral peaks of Markers XIII and XIV are designated within the spectra with a vertical line at the specified molecular weight, i.e. the peak of Marker XIII is shown at the left of the spectra and the peak of Marker XIV is shown at the right of the spectra.

FIG. 14 shows the mass spectra of Marker XV from WCX F6. The mass spectral peak of Marker XV is designated within the spectra with a vertical line at the left of the spectra.

FIG. 15 shows the mass spectra of Marker XVI from IMAC F1. The mass spectral peak of Marker XVI is designated within the spectra with a vertical line.

FIG. 16 shows the mass spectra of Marker XVII from IMAC F1. The mass spectral peak of Marker XVII is designated within the spectra with a vertical line.

FIG. 17 shows the mass spectra of Marker XVIII from IMAC F1. The mass spectral peak of Marker XVIII is designated within the spectra with a vertical line.

FIG. 18 shows the mass spectra of Marker XIX from IMAC F6. The mass spectral peak of Marker XIX is designated within the spectra with a vertical line.

FIG. 19 shows the mass spectra of Marker XX from IMAC F1 and Marker XXIII from IMAC F1. The mass spectral peaks of Markers XX and XXIII are designated within the spectra with a vertical line at the specified molecular weight, i.e. the peak of Marker XX is shown at the right of the spectra and the peak of Marker XXIII is shown at the left of the spectra.

FIG. 20 shows the mass spectra of Marker XXI from IMAC F1 and Marker XXII from IMAC F1. The mass spectral peaks of Markers XXI and XXII are designated within the spectra with a vertical line at the specified molecular weight, i.e. the peak of Marker XXI is shown at the left of the spectra and the peak of Marker XXII is shown at the right of the spectra.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “diseases of the pancreas” or “pancreatic disease,” or “condition of the pancreas,” as used herein, refer to any disease or condition of the pancreatic including, but not limited to, pancreatitis and cancer.

As used herein, “pancreatic cancer,” as used herein, refers to any malignant disease of the pancreas including, but not limited to, adenocarcinoma, small cell undifferentiated carcinoma and mucinous (colloid) cancer.

The term “IMAC F1” refers the biomarkers that are isolated from fraction 1 placed on an IMAC chip”.

The term “WCX F1” refers the biomarkers that are isolated from fraction 1 placed on an WCX chip”.

The term “WCX F6” refers the biomarkers that are isolated from fraction 6 placed on an WCX chip”.

As used herein, “tumor stage” or “tumor progression” refers to the different clinical stages of the tumor. Clinical stages of a tumor are defined by various parameters which are well-established in the field of medicine. Some of the parameters include morphology, size of tumor, the degree in which it has metastasized through the patient's body and the like.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Solid support” refers to a solid material which can be derivatized with, or otherwise attached to, a capture reagent. Exemplary solid supports include probes, microtiter plates and chromatographic resins.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application Ser. No. 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, CM-10, IMAC-3, IMAC-30, LSAX-30, LWCX-30, IMAC40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application Ser. No. US20030032043A1 (Pohl and Papanu, “Latex Based Adsorbent Chip,” Jul. 16, 2002) and U.S. patent application Ser. No. 60/350,110 (Um et al., “Hydrophobic Surface Chip,” Nov. 8, 2001).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Marker” in the context of the present invention refers to a polypeptide (of a particular apparent molecular weight), which is differentially present in a sample taken from patients having human cancer as compared to a comparable sample taken from control subjects (e.g., a person with a negative diagnosis or undetectable cancer, normal or healthy subject). The term “biomarker” is used interchangeably with the term “marker.”

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

“Detect” refers to identifying the presence, absence or amount of the object to be detected.

The phrase “differentially present” refers to differences in the quantity and/or the frequency of a marker present in a sample taken from patients having human cancer as compared to a control subject. For example, some markers described herein are present at an elevated level in samples of cancer patients compared to samples from control subjects. In contrast, other markers described herein are present at a decreased level in samples of cancer patients compared to samples from control subjects. Furthermore, a marker can be a polypeptide, which is detected at a higher frequency or at a lower frequency in samples of human cancer patients compared to samples of control subjects. A marker can be differentially present in terms of quantity, frequency or both.

A polypeptide is differentially present between two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between two sets of samples if the frequency of detecting the polypeptide in the pancreatic cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., pancreatic cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in, the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of pancreatic cancer. A diagnostic amount can be either in absolute amount (e.g., μg/ml) or a relative amount (e.g., relative intensity of signals).

A “control amount” of a marker can be any amount or a range of amount, which is to be compared against a test amount of a marker. For example, a control amount of a marker can be the amount of a marker in a person without pancreatic cancer. A control amount can be either in absolute amount (e.g., μg/ml) or a relative amount (e.g., relative intensity of signals).

As used herein, the term “sensitivity” is the percentage of patients with a particular disease. For example, in the PCa/HC group, the biomarkers of the invention have a sensitivity of about 98%. The panel of biomarkers correctly classified 101 out of 103 pancreatic cancer patients as having pancreatic cancer, i.e. 101/103=98%.

As used herein, the term “specificity” is the percentage of patients correctly identified as having a particular disease i.e. normal or healthy subjects. For example, the specificity is calculated as the number of subjects with a particular disease as compared to normal healthy subjects.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Immunoassay” is an assay that uses an antibody to specifically bind an antigen (e.g., a marker). The immunoassay is characterized by the use of specific binding properties of a particular antibody to isolate, target, and/or quantify the antigen.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Managing subject treatment” refers to the behavior of the clinician or physician subsequent to the determination of pancreatic cancer status. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the status is negative, e.g., late stage pancreatic cancer or if the status is acute, no further action may be warranted. Furthermore, if the results show that treatment has been successful, no further management may be necessary.

### DETAILED DESCRIPTION OF THE INVENTION

The present invention relates to a method for identification of tumor biomarkers markers for pancreatic cancer, with high specificity and sensitivity. In particular, a panel of biomarkers were identified that are associated with pancreatic cancer disease status. We analyzed serum samples from 60 patients with resectable pancreatic adenocarcinoma, and 60 age and sex matched patients with non-malignant pancreatic diseases as well as 60 age and sex matched healthy controls. To increase the number of proteins potentially identifiable, serum was fractionated using anion exchange and profiled on 2 ProteinChip surfaces (IMAC-Cu⁺⁺ and WCX). We determined a minimum set of protein peaks able to discriminate between patient groups and used the software package ProPeak to compare the performance of the individual marker panels alone or in conjunction with CA19-9. Among the many peaks identified by SELDI profiling that had the ability to distinguish between patient groups, the two most discriminating protein peaks were able to differentiate patients with pancreatic cancer from healthy controls with a sensitivity of 93% and specificity of 85%. These 2 markers performed significantly better than the current standard serum marker, CA19-9 (p<0.05). The diagnostic accuracy of these 2 markers was improved by using them in combination with CA 19-9. Similarly, a combination of 3 SELDI markers and CA19-9 was superior to CA19-9 alone in distinguishing individuals with pancreatic cancer from both disease controls and healthy subjects combined. SELDI markers were also better able to distinguish patients with pancreatic cancer from those with pancreatitis than was CA19-9 levels. Accurate differentiation of patients with pancreatic cancer from those with other pancreatic diseases and from healthy controls is possible using SELDI profiling of serum.

I. Description of the Biomarkers

The corresponding proteins or fragments of proteins for these biomarkers are represented as intensity peaks in SELDI (surface enhanced laser desorption/ionization) protein chip/mass spectra with molecular masses centered around the values indicated as follows.

Biomarkers from the WCX F1 biochip include the biomarkers identified as:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD and
Marker VIII: having a molecular weight of about 6.081 kD.

Biomarkers from the WCX F6 biochip include the biomarkers identified as:
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD.

Biomarkers from the IMAC F1 biochip include the biomarkers identified as:
Marker XVI: having a molecular weight of about 4.277 kD
Marker XVII: having a molecular weight of about 4.639 kD
Marker XIII : having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD.

The masses for Markers I-XXXII are considered accurate to within 0.15 percent of the specified value as determined by the disclosed SELDI-mass spectroscopy protocol.

As discussed above, Markers I-XXXII also may be characterized based on affinity for an adsorbent, particularly binding to an immobilized chelate (IMAC)-copper substrate surface or WCX surface under the conditions specified in the Examples, which follow.

The above-identified biomarkers, are examples of biomarkers, as determined by molecular weights, identified by the methods of the invention and serve merely as an illustrative example and are not meant to limit the invention in any way.

A major advantage of identification of these markers is their high specificity and ability to differentiate between different pancreatic disease states. Most pancreatic cancer patients have no known risk factors for tumor development or rate of disease progression. The markers of the invention are therefore important in monitoring and diagnosing for pancreatic cancer progression and to identify patients who are at risk for aggressive disease and would benefit from early treatment.

More specifically, the present invention is based upon the discovery of protein markers that are differentially present in samples of human cancer patients and control subjects, and the application of this discovery in methods and kits for aiding a human cancer diagnosis. Some of these protein markers are found at an elevated level and/or more frequently in samples from human cancer patients compared to a control (e.g., men in whom human cancer is undetectable). Accordingly, the amount of one or more markers found in a test sample compared to a control, or the mere detection of one or more markers in the test sample provides useful information regarding probability of whether a subject being tested has human cancer or not.

The protein markers of the present invention have a number of other uses. For example, the markers can be used to screen for compounds that modulate the expression of the markers in vitro or in vivo, which compounds in turn may be useful in treating or preventing human cancer in patients. In another example, markers can be used to monitor responses to certain treatments of human cancer. In yet another example, the markers can be used in the heredity studies. For instance, certain markers may be genetically linked. This can be determined by, e.g., analyzing samples from a population of human cancer patients whose families have a history of human cancer. The results can then be compared with data obtained from, e.g., human cancer patients whose families do not have a history of human cancer. The markers that are genetically linked may be used as a tool to determine if a subject whose family has a history of human cancer is pre-disposed to having human cancer.

In another aspect, the invention provides methods for detecting markers which are differentially present in the samples of a human cancer patient and a control (e.g., men in whom human cancer is undetectable). The markers can be detected in a number of biological samples. The sample is preferably a biological fluid sample. Examples of a biological fluid sample useful in this invention include blood, blood serum, plasma, nipple aspirate, urine, tears, saliva, etc. Because all of the markers are found in blood serum, blood serum is a preferred sample source for embodiments of the invention.

In accordance with the present invention, the methods described herein, pre-invasive or even benign tumors may be diagnosed by identifying the biomarkers which cause a pre-invasive tumor to progress to a malignant tumor. The type of biomarkers identified and amounts of biomarker may correlate with the jump from a pre-invasive tumor to a malignant stage tumor. Therapy such as immediate excision of the tumor or therapies such as chemotherapy or radiation therapy can be implemented prior to the tumor becoming invasive. The identification of the pre-invasive biomarkers can be used in diagnosis with conventional methods such as, for example, in pancreatic cancer, use of a digital rectal examination.

Any suitable methods can be used to detect one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

The following example is illustrative of the methods used to identify biomarkers for detection of pancreatic diseases. It is not meant to limit or construe the invention in any way. A sample, such as for example, serum from a subject or patient, is immobilized on a biochip. Preferably, the biochip comprises a functionalized, cross-linked polymer in the form of a hydrogel physically attached to the surface of the biochip or covalently attached through a silane to the surface of the biochip. However, any biochip which can bind samples from subjects can be used. The surfaces of the biochips are comprised of, for example, hydrophilic adsorbent to capture hydrophilic proteins (e.g. silicon oxide); carboimidizole-functional groups that can react with groups on proteins for covalent binding; epoxide functional groups for covalent binding with proteins (e.g. antibodies, receptors, lectins, heparin, Protein A, biotin/streptavidin and the like); anionic exchange groups; cation exchange groups; metal chelators and the like.

Preferably, samples are pre-fractionated prior to immobilization as discussed below. Analytes or samples captured on the surface of a biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Immobilized samples or analytes are preferably subjected to laser ionization and the intensity of signal for mass/charge ratio is detected. The data obtained from the mass/charge ratio signal is transformed into data which is read by any type of computer. An algorithm is executed by the computer user that classifies the data according to user input parameters, for detecting signals that represent biomarkers present in, for example, pancreatic cancer patients and are lacking in non-cancer subject controls. The biomarkers are most preferably identified by their molecular weights.

II. Test Samples

A) Subject Types

Samples are collected from subjects who want to establish pancreatic cancer status. The subjects may be men who have been determined to have a high risk of pancreatic cancer based on their family history. Other patients include men and women who have pancreatic cancer and the test is being used to determine the effectiveness of therapy or treatment they are receiving. Also, patients could include healthy people who are having a test as part of a routine examination, or to establish baseline levels of the biomarkers. Samples may be collected from people who had been diagnosed with pancreatic cancer and received treatment to eliminate the cancer, or perhaps are in remission.

B) Types of Sample snd Preparation of the Sample

The markers can be measured in different types of biological samples. The sample is preferably a biological fluid sample. Examples of a biological fluid sample useful in this invention include blood, blood plasma, serum, urine, tissue, cells, organs and seminal fluids, etc. Because all of the markers are found in blood serum, blood serum is a preferred sample source for embodiments of the invention.

If desired, the sample can be prepared to enhance detectability of the markers. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. For example, a K30 spin column (available from Princeton Separation, Ciphergen Biosystems, Inc., etc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by gas phase ion spectrometry for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by removing proteins that are present in a high quantity or that may interfere with the detection of markers in a sample. For example, in a blood serum sample, serum albumin is present in a high quantity and may obscure the analysis of markers. Thus, a blood serum sample can be pre-fractionated by removing serum albumin. Serum albumin can be removed using a substrate that comprises adsorbents that specifically bind serum albumin. For example, a column which comprises, e.g., Cibacron blue agarose (which has a high affinity for serum albumin) or anti-serum albumin antibodies can be used.

In yet another embodiment, a sample can be pre-fractionated by isolating proteins that have a specific characteristic, e.g. are glycosylated. For example, a blood serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Many types of affinity adsorbents exist which are suitable for pre-fractionating blood serum samples. An example of one other type of affinity chromatography available to pre-fractionate a sample is a single stranded DNA spin column. These columns bind proteins which are basic or positively charged. Bound proteins are then eluted from the column using eluants containing denaturants or high pH.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Markers that do not bind to the first adsorbent is removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures.

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot can be is further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described in detail below.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described in detail below.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion fuinction as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

III. Capture of Markers

Biomarkers are preferably captured with capture reagents immobilized to a solid support, such as any biochip described herein, a multiwell microtiter plate, a resin, or nitrocellulose membranes that are subsequently probed for the presence of proteins. In particular, the biomarkers of this invention are preferably captured on SELDI protein biochips. Capture can be on a chromatographic surface or a biospecific surface. Any of the SELDI protein biochips comprising reactive surfaces can be used to capture and detect the biomarkers of this invention. However, the biomarkers of this invention bind well to immobilized metal chelates. The IMAC-3 and IMAC 30 biochips, which nitriloacetic acid functionalities that adsorb transition metal ions, such as Cu⁺⁺ and Ni⁺⁺, by chelation, are the preferred SELDI biochips for capturing the biomarkers of this invention. Other useful BioChips include WCX chips. Any of the SELDI protein biochips comprising reactive surfaces can be used to capture and detect the biomarkers of this invention. These biochips can be derivatized with the antibodies that specifically capture the biomarkers, or they can be derivatized with capture reagents, such as protein A or protein G that bind immunoglobulins. Then the biomarkers can be captured in solution using specific antibodies and the captured markers isolated on chip through the capture reagent.

In general, a sample containing the biomarkers, such as serum, is placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash. The retained protein biomarkers now can be detected by appropriate means.

IV. Detection and Measurement of Markers

Once captured on a substrate, e.g., biochip or antibody, any suitable method can be used to measure a marker or markers in a sample. For example, markers can be detected and/or measured by a variety of detection methods including for example, gas phase ion spectrometry methods, optical methods, electrochemical methods, atomic force microscopy, radio frequency methods, surface plasmon resonance, ellipsometry and atomic force microscopy.

A) SELDI

One preferred method of detection and/or measurement of the biomarkers uses mass spectrometry and, in particular, “Surface-enhanced laser desorption/ionization” or “SELDI”. SELDI refers to a method of desorption/ionization gas phase ion spectrometry (e.g., mass spectrometry) in which the analyte is captured on the surface of a SELDI probe that engages the probe interface. In “SELDI MS,” the gas phase ion spectrometer is a mass spectrometer. SELDI technology is described in more detail above and as follows.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has a only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

B) Immunoassay

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

V. Use of Modified Froms of a Biomarker in Diagnostic Methods

It has been found that proteins frequently exist in a sample in a plurality of different forms characterized by a detectably different mass. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention (including any of Markers I through XXXII) also may be used, themselves, as biomarkers. In certain cases the modified forms may exhibit better discriminatory power in diagnosis than the specific forms set forth herein.

Modified forms of a biomarker including any of Markers I through XXXII can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker and modified forms of it. This method also will also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified from can be accomplished by any specific detection methodology including affinity capture followed by mass spectrometry, or traditional immunoassay directed specifically the modified form. Immunoassay requires biospecific capture reagents, such as antibodies, to capture the analytes. Furthermore, if the assay must be designed to specifically distinguish protein and modified forms of protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

VI. Data Analysis

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., men in whom human cancer is undetectable).

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In preferred methods of the present invention, multiple biomarkers are measured. The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of clinical proteomics for predictive medicine. Subtle variations in data from clinical samples, e.g., obtained using SELDI, indicate that certain patterns of protein expression can predict phenotypes such as the presence or absence of a certain disease, a particular stage of cancer progression, or a positive or adverse response to drug treatments.

Data generation in mass spectrometry begins with the detection of ions by an ion detector as described above. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

In one example, Ciphergen's Biomarker Patterns™ Software is used to detect a pattern in the spectra that are generated. The data is classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

The spectra that are generated in embodiments of the invention can be classified using a pattern recognition process that uses a classification model. In some embodiments, data derived from the spectra (e.g., mass spectra or time-of-flight spectra) that are generated using samples such as “known samples” can then be used to “train” a classification model. A “known sample” is a sample that is pre-classified (e.g., cancer or not cancer). Data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” in any suitable manner. For example, signals above a predetermined signal-to-noise ratio can be selected so that a subset of peaks in a spectrum is selected, rather than selecting all peaks in a spectrum. In another example, a predetermined number of peak “clusters” at a common value (e.g., a particular time-of-flight value or mass-to-charge ratio value) can be used to select peaks. Illustratively, if a peak at a given mass-to-charge ratio is in less than 50% of the mass spectra in a group of mass spectra, then the peak at that mass-to-charge ratio can be omitted from the training data set. Pre-processing steps such as these can be used to reduce the amount of data that is used to train the classification model.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002/0193950 A1 (Gavin et al., “Method or analyzing mass spectra,” Dec. 19, 2002); U.S. 2003/0004402 A1 (Hitt et al., “Process for discriminating between biological states based on hidden patterns from biological data,” Jan. 2, 2003); and U.S. 2003/ 0055615 A1 (Zhang and Zhang, “Systems and methods for processing biological expression data” Mar. 20, 2003).

More specifically, to obtain the biomarkers the peak intensity data of samples from cancer patients and healthy controls are used as a “discovery set.” This data were combined and randomly divided into a training set and a test set to construct and test multivariate predictive models using a non-linear version of Unified Maximum Separability Analysis (“USMA”) classifiers. Details of USMA classifiers are described in U.S. 2003/0055615 A1.

Generally, the data generated from Section IV above is inputted into a diagnostic algorithm (i.e., classification algorithm as described above). The classification algorithm is then generated based on the learning algorithm. The process involves developing an algorithm that can generate the classification algorithm. The methods of the present invention generate a more accurate classification algorithm by accessing a number of pancreatic cancer and normal samples of a sufficient number based on statistical sample calculations. The samples are used as a training set of data on learning algorithm.

The generation of the classification, i.e., diagnostic, algorithm is dependent upon the assay protocol used to analyze samples and generate the data obtained in Section IV above. It is imperative that the protocol for the detection and/or measurement of the markers (e.g., in step IV) must be the same as that used to obtain the data used for developing the classification algorithm. The assay conditions, which must be maintained throughout the training and classification systems include chip type and mass spectrometer parameters, as well as general protocols for sample preparation and testing. If the protocol for the detection and/or measurement of the markers (step IV) is changed, the learning algorithm and classification algorithm must also change. Similarly, if the learning algorithm and classification algorithm change, then the protocol for the detection and/or measurement of markers (step IV) must also change to be consistent with that used to generate classification algorithm. Development of a new classification model would require accessing a sufficient number of pancreatic cancer and normal samples, developing a new training set of data based on a new detection protocol, generating a new classification algorithm using the data and finally, verifying the classification algorithm with a multi-site study.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer. If it is separate from the mass spectrometer, the data must be inputted into the computer by some other means, whether manually or automated.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

VII. Examples of Preferred Embodiments.

The invention provides methods for aiding a human cancer diagnosis using one or more markers, for example Markers in Tables 14 below. These markers can be used alone, in combination with other markers in any set, or with entirely different markers in aiding human cancer diagnosis. The markers are differentially present in samples of a human cancer patient, for example pancreatic cancer patient, and a normal subject in whom human cancer is undetectable. For example, some of the markers are expressed at an elevated level and/or are present at a higher frequency in human cancer patients than in normal subjects. Therefore, detection of one or more of these markers in a person would provide useful information regarding the probability that the person may have human cancer.

In a preferred embodiment, a serum sample is collected from a patient and then fractionated using an anion exchange resin as described above. The biomarkers in the sample are captured using an IMAC3 Cu++ ProteinChip array or WCX chip. The markers are then detected using SELDI. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

The diagnosis is determined by examining the data produced from the SELDI tests with the classification algorithm that is developed using the biomarkers. The classification algorithm depends on the particulars of the test protocol used to detect the biomarkers. These particulars include, for example, sample preparation, chip type and mass spectrometer parameters. If the test parameters change, the algorithm must change. Similarly, if the algorithm changes, the test protocol must change.

In another embodiment, the sample is collected from the patient. The biomarkers are captured using an antibody ProteinChip array as described above. The markers are detected using a biospecific SELDI test system. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

In yet other preferred embodiments, the markers are captured and tested using non-SELDI formats. In one example, the sample is collected from the patient. The biomarkers are captured on a substrate using other known means, e.g., antibodies to the markers. The markers are detected using methods known in the art, e.g., optical methods and refractive index. Examples of optical methods include detection of fluorescence, e.g., ELISA. Examples of refractive index include surface plasmon resonance. The results for the markers are then subjected to an algorithm, which may or may not require artificial intelligence. The algorithm produces a diagnosis based upon the data received relating to each biomarker.

In any of the above methods, the data from the sample may be fed directly from the detection means into a computer containing the diagnostic algorithm. Alternatively, the data obtained can be fed manually, or via an automated means, into a separate computer that contains the diagnostic algorithm. Exemplary Markers of the invention are illustrated in Tables 1 through 4:

TABLE 1
Markers on WCX F1 chip

| | M/Z (Da)^{a} |
|---|---|
| MARKER No. | WCX F1 |
| I | 3667 |
| II | 7451 |
| III | 3144 |
| IV | 12861 |
| V | 3760 |
| VI | 4053 |
| VII | 5884 |
| VIII | 6081 |

^{a}M/Z (mass-dependent velocities)

TABLE 2
Markers on WCX F6 chip

| | M/Z (Da)^{a} |
|---|---|
| MARKER No. | WCX F6 |
| IX | 3473 |
| X | 5903 |
| XI | 8563 |
| XII | 16008 |
| XIII | 4159 |
| XIV | 4179 |
| XV | 7607 |

TABLE 3
Markers on IMAC F1 chip

| | M/Z (Da)^{a} |
|---|---|
| MARKER No. | IMAC F1 |
| XVI | 4277 |
| XVII | 4639 |
| XIII | 6093 |
| XIX | 7463 |
| XX | 9132 |
| XXI | 3885 |
| XXII | 3967 |
| XXIII | 8929 |

TABLE 4
Markers on IMAC Cu⁺⁺ chip

| | M/Z (Da)^{a} |
|---|---|
| MARKER No. | IMAC Cu++ |
| XXIV | 3370 |
| XXV | 3441 |
| XXVI | 10055 |
| XXVII | 3510 |
| XXVIII | 9120 |
| XXIX | 7294 |
| XXX | 8866 |
| XXXI | 9401 |
| XXXII | 8754 |

Accordingly, embodiments of the invention include methods for aiding a human cancer diagnosis, wherein the method comprises: (a) detecting at least one marker in a sample, wherein the marker is selected from any of the Marker in Tables 1 through 3; and (b) correlating the detection of the marker or markers with a probable diagnosis of human cancer. The correlation may take into account the amount of the marker or markers in the sample compared to a control amount of the marker or markers (up or down regulation of the marker or markers) (e.g., in normal subjects in whom human cancer is undetectable). The correlation may take into account the presence or absence of the markers in a test sample and the frequency of detection of the same markers in a control. The correlation may take into account both of such factors to facilitate determination of whether a subject has a human cancer or not.

Any suitable samples can be obtained from a subject to detect markers. Preferably, a sample is a blood serum sample from the subject. If desired, the sample can be prepared as described above to enhance detectability of the markers. For example, to increase the detectability of markers in Tables 1 through 3, a blood serum sample from the subject can be preferably fractionated by, e.g., Cibacron blue agarose chromatography and single stranded DNA affinity chromatography, anion exchange chromatography and the like. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

VIII. Diagnosis of Subject and Determination of Pancreatic Cancer Status

Any biomarker, individually, is useful in aiding in the determination of pancreatic cancer status. First, the selected biomarker is measured in a subject sample using the methods described herein, e.g., capture on a SELDI biochip followed by detection by mass spectrometry. Then, the measurement is compared with a diagnostic amount or control that distinguishes a pancreatic cancer status from a non-cancer status. The diagnostic amount will reflect the information herein that a particular biomarker is up-regulated or down-regulated in a cancer status compared with a non-cancer status. As is well understood in the art, the particular diagnostic amount used can be adjusted to increase sensitivity or specificity of the diagnostic assay depending on the preference of the diagnostician. The test amount as compared with the diagnostic amount thus indicates pancreatic cancer status.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and would decrease the percentage of false positive or false negative diagnoses. Thus, preferred methods of the present invention comprise the measurement of more than one biomarker. For example, the methods of the present invention have an AUC from ROC analysis greater than 0.50, more preferred methods have an AUC greater than 0.60, more preferred methods have an AUC greater than 0.70. Especially preferred methods have an AUC greater than 0.70 and most preferred methods have an AUC greater than 0.80.

In order to use the biomarkers in combination, a logistical regression algorithm is useful. The UMSA algorithm is particularly useful to generate a diagnostic algorithm from test data. This algorithm is disclosed in Z. Zhang et al., Applying classification separability analysis to microaary data. In: Lin S M, Johnson K F, eds. Methods of Microarray data analysis: papers from CAMDA '00. Boston: Kluwer Academic Publishers, 2001:125-136; and Z. Zhang et al., Fishing Expedition—a Supervised Approach to Extract Patterns from a Compendium of Expression Profiles. In Lin S M, Johnson, K F, eds. Microarray Data Analysis II: Papers from CAMDA '01. Boston: Kluwer Academic Publishers, 2002.

The learning algorithm will generate a multivariate classification (diagnostic) algorithm tuned to the particular specificity and sensitivity desired by the operator. The classification algorithm can then be used to determine pancreatic cancer status. The method also involves measuring the selected biomarkers in a subject sample. These measurements are submitted to the classification algorithm. The classification algorithm generates an indicator score that indicates cancer status.

The detection of the marker or markers is then correlated with a probable diagnosis of human cancer. In some embodiments, the detection of the mere presence or absence of a marker, without quantifying the amount of marker, is useful and can be correlated with a probable diagnosis of human cancer. For example, markers in Tables 1 through 4 above can be more frequently detected in human pancreatic cancer patients than in normal subjects. A mere detection of one or more of these markers in a subject being tested indicates that the subject has a higher probability of having a human cancer.

In other embodiments, the measurement of markers can involve quantifying the markers to correlate the detection of markers with a probable diagnosis of cancer. Thus, if the amount of the markers detected in a subject being tested is different compared to a control amount (i.e., higher or lower than the control, depending on the marker), then the subject being tested has a higher probability of having cancer.

The correlation may take into account the amount of the marker or markers in the sample compared to a control amount of the marker or markers (up or down regulation of the marker or markers) (e.g., in normal subjects in whom human cancer is undetectable). A control can be, e.g., the average or median amount of marker present in comparable samples of normal subjects in whom human cancer is undetectable. The control amount is measured under the same or substantially similar experimental conditions as in measuring the test amount. The correlation may take into account the presence or absence of the markers in a test sample and the frequency of detection of the same markers in a control. The correlation may take into account both of such factors to facilitate determination of pancreatic cancer status.

In certain embodiments of the methods of qualifying cancer status, the methods further comprise managing subject treatment based on the status. As aforesaid, such management describes the actions of the physician or clinician subsequent to determining cancer status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive chemotherapy or radiation treatments, either in lieu of, or in addition to, surgery. Likewise, if the result is negative, e.g., the status indicates late stage cancer or if the status is otherwise acute, no further action may be warranted. Furthermore, if the results show that treatment has been successful, no further management may be necessary.

The invention also provides for such methods where the biomarkers (or specific combination of biomarkers) are measured again after subject management. In these cases, the methods are used to monitor the status of the cancer, e.g., response to cancer treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

The methods of the present invention have other applications as well. For example, the markers can be used to screen for compounds that modulate the expression of the markers in vitro or in vivo, which compounds in turn may be useful in treating or preventing cancer in patients. In another example, the markers can be used to monitor the response to treatments for cancer. In yet another example, the markers can be used in heredity studies to determine if the subject is at risk for developing cancer. For instance, certain markers may be genetically linked. This can be determined by, e.g., analyzing samples from a population of pancreatic cancer patients whose families have a history of pancreatic cancer. The results can then be compared with data obtained from, e.g., cancer patients whose families do not have a history of pancreatic cancer. The markers that are genetically linked may be used as a tool to determine if a subject whose family has a history of pancreatic cancer is pre-disposed to having pancreatic cancer.

IX. Kits

In yet another aspect, the invention provides kits for aiding a diagnosis of human cancer, wherein the kits can be used to detect the markers of the present invention. For example, the kits can be used to detect any one or more of the markers described herein, which markers are differentially present in samples of a human cancer patient and normal subjects. The kits of the invention have many applications. For example, the kits can be used to differentiate if a subject has human pancreatic cancer or has a negative diagnosis, thus aiding a human cancer diagnosis. In another example, the kits can be used to identify compounds that modulate expression of one or more of the markers in in vitro or in vivo animal models for human cancer.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample of blood serum is contacted on the probe. In another example, the kit may have instructions for pre-fractionating a sample to reduce complexity of proteins in the sample. In another example, the kit may have instructions for automating the fractionation or other processes.

In another embodiment, a kit comprises (a) an antibody that specifically.binds to a marker; and (b) a detection reagent. Such kits can be prepared from the materials described above, and the previous discussion regarding the materials (e.g. antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of human pancreatic cancer.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

### Materials and Methods

SELDI

Surface enhanced-laser desorption /ionization is an affinity-based mass spectrometry method in which proteins of interest are selectively adsorbed to a chemically modified surface on the chip. Other substances are removed by washing steps.

Materials and Methods:

Patients and samples: A total of 180 patients from the Johns Hopkins Medical Institutions were studied. All samples were collected and analyzed with approval from the Johns Hopkins Committee for Clinical Investigation. Preoperative blood was collected from 60 patients undergoing pancreaticoduodenectomy for pancreatic adenocarcinoma. The disease control group consisted of 60 age and sex matched patients with pancreatic disease who were undergoing pancreaticoduodenectomy (Whipple procedure) or endoscopic ultrasound-guided fine needle aspiration at the Johns Hopkins Hospital for suspected pancreatic cancer or peripancreatic disease. The disease control subgroup consisted of patients with pancreatitis (n=26), neuroendocrine tumors (n=8), pancreatic cysts (n=8), pancreatic cystadenoma (n=6), ampullary adenoma (n=4), intraductal papillary mucinous neoplasms (IPMN) (n=4), low-grade pancreatic intraepithelial neoplasia (PanIN) (n=2), duodenal adenoma (n=1) and choledochal cyst (n=1). Histopathologic diagnosis was available on all patients with pancreatic cancer and for 30 of the 60 patients in the disease control specimens. In the remaining cases diagnosis was based on cytology or clinical information. The subgroup of normal controls consisted of 60 age and sex matched individuals without known malignant disease taking part in a longitudinal study of aging. In those patients with pancreatic cancer, all blood samples were collected preoperatively. Samples were collected between 1997 and 2002, and were samples were stored at −80° C. for all subgroups. The mean age of the groups were normal controls 64.8±10.5, disease controls 61.9±7.9, pancreatic cancer 64.1±8.4 years). Each patient group contained 30 female and 30 males.

SELDI ProteinChip analysis: For the SELDI ProteinChip analysis, two biochemically distinct chip surfaces, the IMAC-Cu⁺⁺ (immobilized metal affinity capture) and WCX (weak cation exchange) chips, were chosen to increase the proportion of the serum proteome represented on chip for mass spectrometric analysis. To further improve protein peak yield, an anion exchange fractionation procedure was performed in which serum was separated into six different fractions (ph9+flowthrough, pH7, pH5, pH4, pH3, organic wash) prior to on chip analysis. This fractionation procedure significantly increases the number of peaks detectable from each individual serum sample (19). Each sample fraction was randomly assigned to a spot in a 192 spot format on 24 protein chips that included the 180 patients as well as twelve aliquots of a pooled human serum sample (Intergen Corp.) for quality control purposes. Each fraction was analyzed in duplicate.

For the anion exchange fractionation, 30 μl of U9 buffer (9M, Urea, 2% CHAPS, 50 mM Tris-HCl, pH9) was added to 20 μl of each serum sample and vortexed at 4° C. for 20 min. HyperQ DF resin (BioSepra SA) was prepared by washing three times with 5 bed volumes of 50 mM Tris-HCl, pH9. 180 μl of the resin suspension was then aliquoted on a 96 well filter plate (Greiner Corp.) and equilibrated by washing three times with 200 μl of U1 buffer (1M Urea, 0.22% CHAPS, 50 mM Tris-HCl, pH9) on a vacuum manifold (Beckman Coulter Inc.). 50 μl of the serum/U9 mix was then added to the resin in each well of the filter plate. An additional wash of the sample plate with 50 μl of U1 was performed and added to the filter plate. Plates were then vortexed at 4° C. for 30 min to bind the serum to the anion exchange resin. Consecutively, 100 μl of wash buffer was added to each well, vortexed for 10 min. at room temperature and the eluate fraction collected via vacuum manifold. The wash buffers for the different fractions were: 50 mM Tris-HCl, 0.1% OGP, pH9 (F1), 50 mM Hepes, 0.1% OGP, pH7 (F2), 100 mM Na-Acetate, 0.1% OGP, pH5 (F3), 100 mM Na-Acetate, 0.1% OGP, pH4 (F4), 50 mM Na-Citrate, 0.1% OGP, pH 3 (F5) and 33.3% isopropanol/16.7% acetonitrile/0.1% trifluoric acid (F6). All pipetting steps utilized a Biomek 2000 laboratory workstation (Beckman Coulter Inc.). Collected fractions were stored at −80° C. until final analysis.

For ProteinChip binding, IMAC2 chips (Ciphergen Biosystems Inc., Palo Alto, Calif.) were preloaded with 50 μl CuSO₄ (100 mM) per spot on a bioprocessor module (Ciphergen Biosystems Inc.), which allows simultaneous processing of 12 ProteinChips, vortexed for 5 min. and rinsed with H₂Odd. ProteinChips were then equilibrated 2× with 150 μl chip binding buffer (Phosphate buffered saline, pH 7.4 for IMAC2 and 100 mM Na-Acetate pH 4.0 for WCX). Ten μl of the fractionated eluate and 90 μl of the respective binding buffer were then added on each spot and vortexed for 30 min. After discarding the remaining sample, chips were washed 3× with 150 μl of binding buffer and two water rinses. Sinapinic acid solution as energy absorbing matrix (EAM) was prepared according to the manufacturer's instructions (Ciphergen Biosystems Inc.) in 500 ml/L acetonitrile/5ml/L trifluoric acid and 0.5 μl of the saturated solution applied twice to each spot on the chip. ProteinChips were air dried and stored at room temperature in the dark until further use.

All chips were read on a Protein Biological System II reader (Ciphergen Biosystems, Inc.). The high mass setting was set to acquire at 100 kDa, with an optimization range between 3 and 20 kDa. Mass spectrometry profiles were generated by averaging 110 laser shots at two different laser intensities (between 200-280) and detector sensitivities (between 6-10), determined individually for each fraction on the basis of maximum protein peak yield. External calibration of the instrument was performed using the All-in-1 peptide molecular mass standard (Ciphergen Biosystems, Inc.).

CA 19-9 ELISA: 25 μl of serum were analyzed with a commercially available ELISA kit (MucinPC/CA19-9 ELISA, Alpha Diagnostic Int.) according to the manufacturer's recommendations.

Data analysis: Peak detection was performed using the ProteinChip Biomarker software version 3.0 (Ciphergen Biosystems, Inc.). All spectra were compiled, normalized to the total ion current of m/z between 2000 and 100000 and the baselines subtracted. The part of the spectrum with m/z values <2000 was not used for analysis, as the EAM signal generally interfered with peak detection in this area. Peaks between 2000 and 100000 m/z ratios were autodetected with a signal-to-noise-ratio of >5 and the peaks clustered using second-pass peak selection with signal-to-noise >2 and a 0.3% mass window. The resulting peak intensity values were logarithmically transformed to reduce the variance of the data over multiple samples (14).

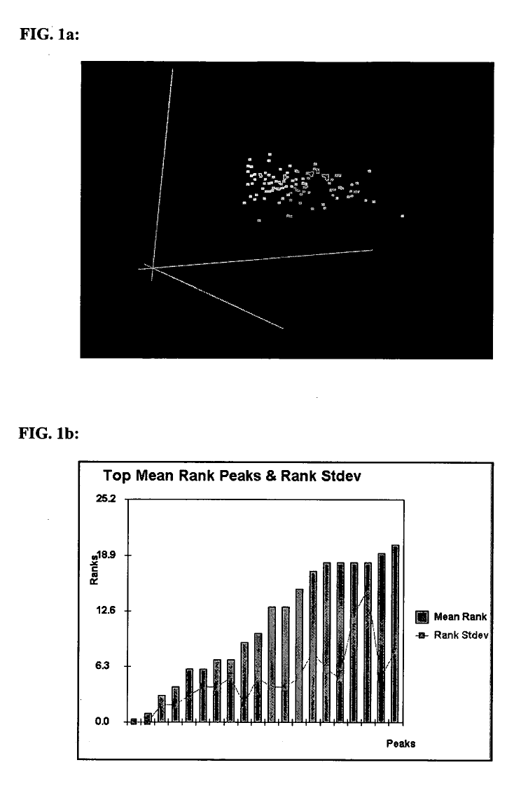

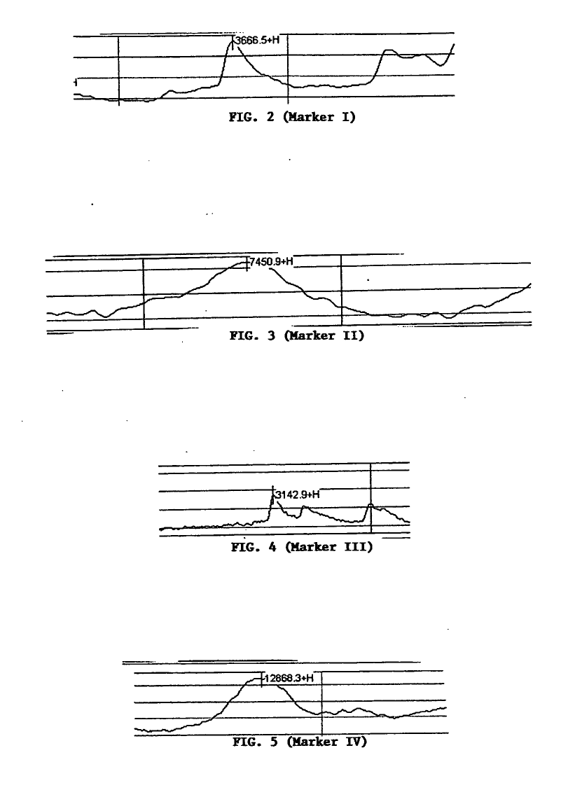

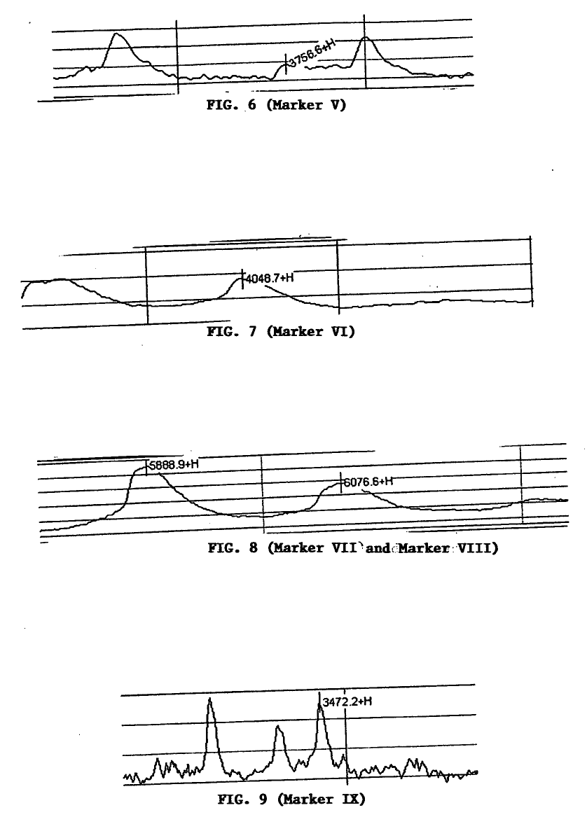

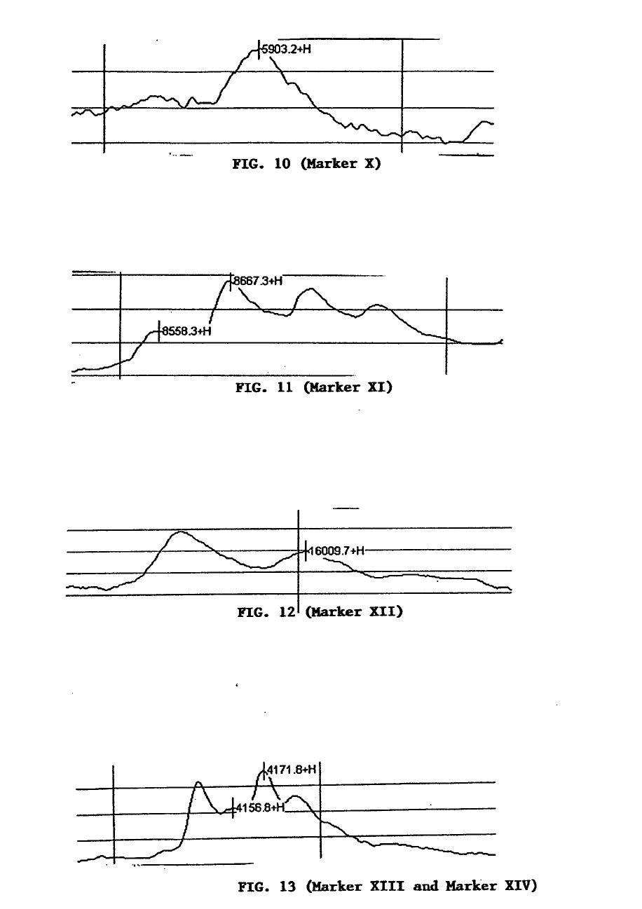

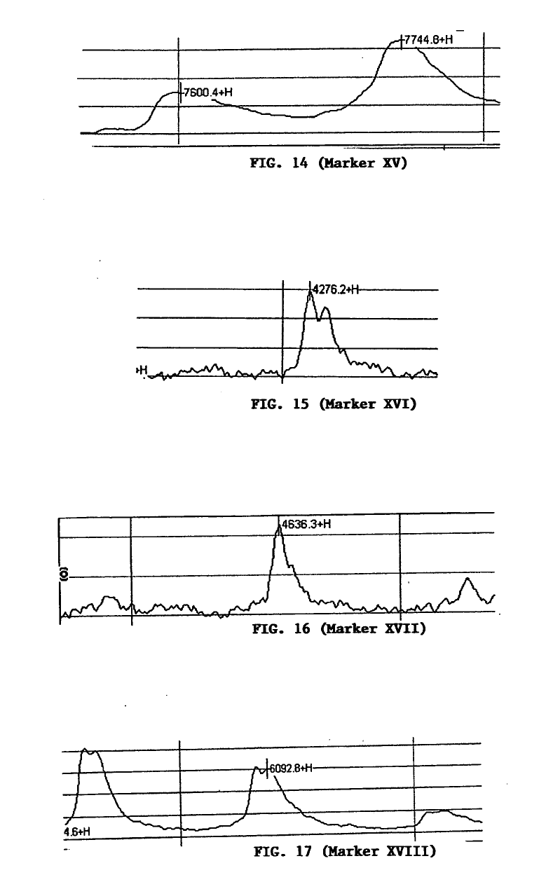

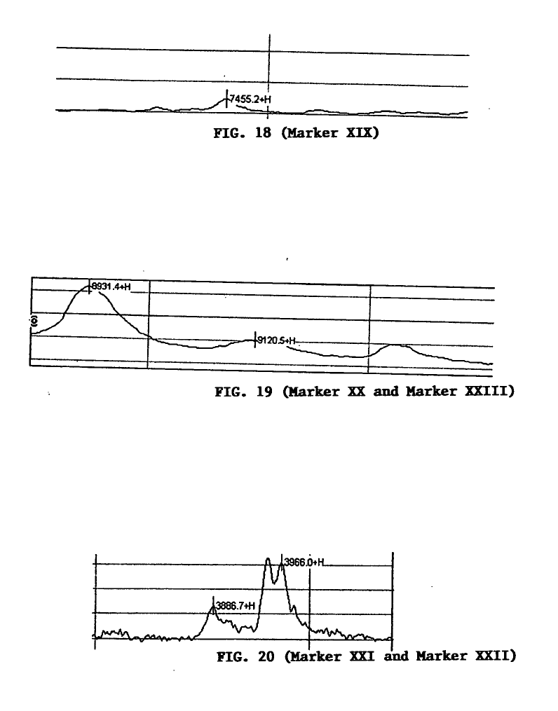

Further analysis of the mass spectrometry data was performed using the ProPeak software package (3Z Informatics) (13, 14). ProPeak implements the linear version of the Unified Maximum Separability Analysis (UMSA) algorithm. This algorithm uses data distribution information to identify a direction along which two predefined sets of data achieve maximum separation. In the first ProPeak module, Component Analysis, each sample is represented in an interactive three-dimensional display (see FIG. 1a). The axes of this coordinate system are linear combinations of peak intensity data. Separability of two data sets can so be assessed visually. Using the second ProPeak module, BootStrap selection, the number of peaks needed for an acceptable separation of the data can be reduced to a minimum by multiple iterations of the UMSA algorithm with a subset of data being omitted randomly (30 iterations with 30% leave-out rate in our study). Thus the relative contribution of each peak to data separation can be displayed as mean, median and corresponding SD of the peak's ranking (see FIG. 1b). To reduce the influence of random variations in peak intensity, each protein peak used in the further analysis had to rank consistently within the top ranks of both replicates of the original sample sets. For the identification of pancreatic cancer biomarkers, we compared the following groups: Pancreatic adenocarcinoma vs. healthy controls, pancreatic adenocarcinoma vs. non-pancreatic cancer cases (all healthy controls and disease controls combined), and pancreatic adenocarcinoma vs. the subgroup of 26 patients with pancreatitis. For each of these comparisons, a panel of peaks (typically 6-12) with high ranking and reproducibility between replicates could be derived. M/Z values that were in the 0.3% mass accuracy window were considered to be identical between replicates. For each of the peaks, its significance for data separation was assessed using mean peak height values and p values from the two-sample t-test. To represent the performance of the multiple marker panels, a composite index was generated by multivariate logistic regression, which enabled the calculation of sensitivity, specificity and receiver-operator-characteristics (ROC) curves using the complete data set.

Results:

Peak detection: A total of 12 fractions (6 fractions each from WCX and IMAC-Cu⁺⁺ surfaces) were read on the PBS II reader. For both chip types fraction 2 (pH7) contained an insufficient number (<20) of protein peaks per sample and was therefore omitted from analysis. The amount of qualified peaks (signal-to-noise-ratio >5) detected in each of the remaining fractions varied between 21-185, with fraction 1 (pH9+flowthrough) yielding the most protein peaks on both chip surfaces (see Table 4). A specific albumin signal was observed only in fractions 3-5 on each chip type, with a corresponding increase in the detection of low-abundance signal in fractions 1 and 6. Most of the peaks detected clustered in the 2-20 kDa range, with only a few peaks detected between 20 and 100 kDa. Generally, the protein spectra of each fraction were unique and complementary to each other, but some peaks were detectable in up to 3 fractions with varying intensity, indicating possible carry-over during the fractionation process.

TABLE 4
M/Z values for the SELDI derived protein peaks used in the diagnostic Panels

| | WCX F1 | WCX F6 | IMAC F1 |
|---|---|---|---|
| Cancer vs. non-cancer | 3667 (Marker I) | 3473 (Marker IX) | 4277 (Marker XVI) |
| | 7451 (Marker II) | 5903 (Marker X) | 4639 (Marker XVII) |
| | 12861 (Marker IV) | 8563 (Marker XI) | 6093 (Marker XIII) |
| | | | 7463 (Marker XIX) |
| | | | 9132 (Marker XX) |
| Cancer vs. normal | 3144 (Marker III) | 3473 (Marker IX) | 3885 ((Marker XXI) |
| | 12861 (Marker IV) | 5903 (Marker X) | 3967 (Marker XXII) |
| | | 8563 (Marker XI) | 8929 (Marker XXIII) |
| | | 16008 (Marker XII) | |
| Cancer vs. pancreatitis | 3760 (MarkerV) | 4159 (MarkerXIII) | 6093 (Marker XVIII) |
| | 4053 (MarkerVI) | 4179 (MarkerXIV) | 7463 (Marker XIX) |
| | 5884 (MarkerVII) | 7607 (MarkerXV) | |
| | 6081 (MarkerVII) | | |

Serum SELDI Profiles of Pancreatic Adenocarcinoma Versus Healthy Controls:

On the WCX ProteinChip surface, a total of 13 peaks in fraction 1 (pH 9+flowthrough) and 12 peaks in fraction 6 (organic wash) could be used to discriminate between serum from patients with pancreatic cancer and that from healthy controls and that of non-cancer controls by means of their reproducibly high ranking on multiple iterations of the UMSA algorithm (see FIG. 1b) on both replicates of the original data set. Analysis of fraction 1 samples with the IMAC-Cu⁺⁺ Surface, yielded 10 usable peaks. Among these peaks, the 2 most discriminating peaks obtained from fraction 1 profiled on the WCX chip (m/z 3144, 12861) and the most discriminating 4 peaks in fraction 6 (m/z 3473, 5903, 8563, 16008) were significantly better at distinguishing between serum from patients with pancreatic adenocarcinoma versus that of healthy controls than was CA19-9 (p<0.05). The respective area-under-the-curve (AUC) for the receiver-operator-characteristics (ROC) curve was 0.96 for the 2-peak panel, 0.97 for the 4-peak panel and 0.85 for CA19-9 (see Table 4). Combining the SELDI protein peaks and CA19-9 yielded a small improvement in the ability to distinguish between those with pancreatic adenocarcinoma and healthy controls: the AUC improved to 0.98 (4 peaks and CA19-9) and 0.99 (2 peaks and CA19-9) (see Table 4), indicating that SELDI derived markers and CA19-9 had some complementary diagnostic utility. The 3 most discriminating markers from the IMAC-^{Cu++} chip profiles (m/z 3885, 3967, 8929) could also distinguish between pancreatic cancer and healthy control with good accuracy but not as effectively as the peaks identified from the WCX chip profiles (AUC 0.86).

Serum SELDI Profiles of Pancreatic Cancers Versus Other Non-Malignant Pancreatic Diseases:

For the comparison of the pancreatic cancer group vs. the non-malignant pancreatic disease controls, 3 peaks (m/z 3667, 7451, 12861) derived from WCX, fraction 1 as well as 3 peaks (m/z 3473, 5903, 8563) from WCX, fraction 6 yielded an AUC of 0.82 and 0.78, respectively. This degree of separation of these groups was not significantly different from that achievable with CA19-9 (AUC 0.80) (see Table 4). However, combining SELDI profiling and CA19-9 could differentiate those with pancreatic cancer from the group with other pancreatic diseases more accurately than Ca19-9 alone. The combination of CA19-9 and the 3-peaks identified from WCX fraction 1) had an AUC of 0.90 (p<0.05). Combining CA19-9 with the 3-peak panel identified from WCX fraction 6 profiling yielded no significant improvement (AUC 0.81). Similar results were obtained using the IMAC-Cu⁺⁺ chip, the top 5 peaks from fractionl (m/z 4277, 4639, 6093, 7463, 9132) achieved an AUC of 0.80 for distinguishing between pancreatic cancer from the group consisting of other pancreatic diseases.

Comparison of SELDI Serum Profiles from Patients with Pancreatic Cancer Versus Pancreatitis:

Since our disease control group included a significant subset of patients with pancreatitis, we conducted a subgroup analysis of pancreatic cancer versus pancreatitis. A panel of four peaks identified from WCX chip profiles of fraction 1 (m/z 3760, 4053, 5884, 6081) could distinguish serum from patients with pancreatic cancer from those with pancreatitis significantly better (p<0.05) than CA19-9 (AUC 0.82 vs. 0.69) (see Table 4). These 4 peaks that were optimal for differentiating pancreatic cancer from pancreatitis were distinct from those distinguished pancreatic cancer from the larger group of disease controls. Adding CA19-9 to these 4-peak was only more accurate than using the SELDI peaks alone (AUC 0.85). Similar results were found from SELDI peaks identified from profiles of WCX chip fraction 6. A 3-peak panel, (m/z values 4159, 4179, 7607), distinct from the aforementioned peaks, was significantly better than CA19-9 (p<0.05) at distinguishing pancreatic cancer from pancreatitis (AUC 0.87 and 0.68). Combining these peaks with Ca19-9 was no better than using these peaks alone. Once again the IMAC-Cu⁺⁺ surface was less sensitive with the 2 most discriminating markers from fraction 1 (m/z 6093, 7463) yielding an AUC of 0.69.

Duodenal Juice SELDI Profiling

Duodenal juice specimens were analyzed from patients with pancreatic cancer, chronic pancreatitis and other non-pancreatic gastrointestinal diseases. Duodenal juice is pancreatic juice, collected via gastroscopy (EGD) after iv. secretin stimulation.

Materials and Methods:

Duodenal juice was collected by aspiration after iv. Secretin application via EGD from 23 pateints with pancreatic cancer, 17 with chronic pancreatitis and 23 with non-pancreatic gastrointestinal diseases (controls). The patients were part of a prospective screening study for pancreatic cancer at the Johns Hopkins Medical Institutions. Unfractionated duodenal juice was analyzed by surface-enhanced laser desorption and ionization (SELDI) on IMAC3-Cu⁺⁺ ProteinChips (Ciphergen) by a standard protocol. Chips were read on a PBS IIC ProteinChip reader and data were analyzed using the ProPeak software package (3Z Informatics). Internal quality control samples consisting of pooled serum and pooled duodenal juice samples were run on each chip. All experiments were performed in duplicate.

Results:

For the differentiation of pancreatic cancer samples from control samples (other GI disease), a consistent set of 3 peaks (m/z values: 3370, 3441, 10055 Da) which retained most of the discriminating value of the total set of 173 peaks was identified on both replicates of the data set. Markers 3370 Da and 3441 Da were upregulated in pancreatic cancer samples; marker 10055 Da downregulated. For the differential diagnosis of pancreatic cancer from chronic pancreatitis, a larger number of peaks (m/z values: 3510, 9120, 7294, 8866, 9401, 8754) exhibited diagnostic potential.

In the overall differentiation of cancer and non-cancer (chronic pancreatitis and other GI diseases combined) samples, the 3370 Da, 3441 Da, 3510Da and 10055 Da markers ranked best.

The above results show that SELDI profiling of serum is significantly better than CA19-9 at distinguishing patients with pancreatic cancer from those with pancreatitis and from healthy controls. The superiority of SELDI was evident over multiple serum fractions and multiple chip types. In addition, when used in combination with Ca19-9, SELDI could more accurately differentiate patients with non-malignant pancreatic disease than Ca19-9 alone. Importantly since most of the patients with pancreatic cancer had small surgically resectable cancers, it is likely that the markers we have identified using SELDI will be diagnostically useful for the patients that are hardest to diagnose, those with small cancers. We chose to include patients with a variety of pancreatic diseases in our disease control group in order to best mimic real life diagnostic difficulties. Since this group included patients with benign neoplastic pancreatic diseases such as intraductal papillary mucinous neoplasms, it is not surprising that SELDI profiling had somewhat less accuracy in differentiating pancreatic cancer from the group containing a variety of pancreatic diseases than it was for a more homogeneous group of patients with pancreatitis or from healthy controls. We also wanted to include a heterogeneous disease control group as some of the biomarkers discovered in serum by SELDI-based methods have been inflammatory in nature reflecting cancer-induced, non-specific tissue injury (13) and inclusion of disease control helps to differentiate markers that are inflammatory in nature from true cancer-specific molecules.

Pancreatic cancer induces increases in a variety of serum markers including proteins derived from the neoplastic cells in the case of CA19-9 and others, from surrounding acini in the case of HIP, as well as from surrounding stroma that could include inflammatory, or matrix markers (18, 22). Since we found that different protein peaks were needed to differentiate between pancreatic cancer sera versus healthy control sera and pancreatic cancer sera vs. pancreatitis sera, and pancreatic cancer vs. disease control sera, we suspect that for each comparison the proteins that distinguish between these groups could arise from different sources. For example, the proteins that distinguish pancreatic cancer sera from pancreatitis sera are less likely to be inflammatory or acinar proteins than those that differentiate pancreatic cancer sera from healthy control sera.

Our SELDI protocol may have facilitated the identification of novel proteins. First, we used anion exchange fractionation to increase the total number of peaks detectable in an individual sample. One way fractionation improves SELDI resolution is that it removes the most abundant serum protein, albumin, to which many low-abundance proteins bind, in fractions 1 (pH9+flowthrough) and 6 (organic wash), thus enabling the detection of more low-abundance proteins in these fractions. These steps may have been one reason why we were able to distinguish with reasonable accuracy serum from patients with pancreatic cancer versus those with pancreatitis, unlike a previous matrix associated laser desorption ionization (MALDI) mass spectrometry study (23).

One of the challenges in the analysis of SELDI mass spectrometry-generated data is avoiding the false discovery of protein peaks, whose discriminatory power is due to random variation. The UMSA algorithm used to analyze the SELDI profiles in this study reduces this problem by ranking all detected protein peaks according to their relative contribution to the separation of distinct data sets, and by using Boot Strap cross-validation which involves the random omission of a specified amount of peaks for multiple iterations of analysis. As a further safeguard against the identification of discriminating peaks that are merely artifacts, we analyzed all samples in duplicate, and only peaks which exhibited a reproducibly high ranking in both sets of analysis were used for further analysis to identify the most discriminating peaks. This approach allowed us to define a minimum set of protein peaks (typically 2-4 peaks) needed to distinguish between patient groups with the most discriminatory power. Indeed, the number of peaks needed to discriminate between patient groups in our study was fewer than have been needed in previous SELDI studies involving other cancers (12, 16, 24).

Although SELDI profiling alone may permit accurate diagnosis without identification of protein peak identity, the identification of a limited number of protein peaks necessary for accurate SELDI-based diagnosis of pancreatic cancer raises the possibility that these proteins can be purified and identified facilitating the development of an antibody-based clinical test. Further characterization of SELDI protein peaks can be done using ProteinChip surfaces with chromatography capability to facilitate the separation and identification of diagnostically useful protein and peptides within complex biological samples (10, 19).(13)

SELDI proteomic profiles have been reported to exhibit a high degree of diagnostic accuracy in the serologic diagnosis of ovarian, pancreatic and breast cancer (12-14, 16, 24). The range of the relevant protein peaks detected in those studies is comparable to our study (2-20 kDa), with the exception of the initial report of SELDI data in the diagnosis of ovarian cancer (0.5-2.4 kDa). As to the protein identity of the m/z peaks observed in our study, it is notable that they were distinct from the 3 SELDI derived peaks, identified by Rai et.al. as transferrin, haptoglobin precursor protein and immunoglobulin heavy chain in ovarian cancer sera (13). Our results as well as those of other investigators studying other cancers suggest that future cancer diagnosis will require separate marker panels depending on whether the clinical question being asked is differentiating cancer from inflammatory conditions or perhaps screening healthy individuals for the possible presence of cancer. The need for multiple markers for cancer diagnosis is not surprising given the biological heterogeneity of cancer. In addition, our serum profiling did not identify a peak at 16570 D, corresponding to HIP/PAP which was previously identified by profiling pancreatic juice (18). This observation illustrates the limits of serum protein diagnostics, as some cancer markers being more likely to be released locally than to be secreted into the general circulation. SELDI profiling of pancreatic juice obtained during upper endoscopy after secretin stimulation may also have diagnostic utility and may be especially helpful for diagnosing small lesions such as benign neoplasms of the pancreas which may not lead to as many changes in serum proteins as seen with pancreatic cancer. In addition, current SELDI protocols fail to resolve many serum proteins as evidenced in this study by the inability to detect other markers elevated in the serum of patients with pancreatic cancer such as HIP or CA19-9.

Proteomics approaches complement global gene expression approaches, which are powerful tools for identifying differentially expressed genes but are hampered by the imperfect correlation of MRNA levels and protein and by the limitation that only a small number of differentially expressed genes are secreted proteins whose serum levels will be altered by disease (25).

The methods of the present invention demonstrate that SELDI profiling of serum is not only be useful for diagnosing patients who present with pancreatic disease but is also useful for screening individuals at high-risk for the development of pancreatic cancer. Currently, there is no validated screening strategy for high risk individuals. At our institution patients enrolled in the National Familial Pancreatic Tumor Registry (NFPTR) who have a strong family history of pancreatic cancer and other groups with a high lifetime risk of pancreatic cancer such as those with Peutz-Jeghers syndrome can undergo a pilot screening protocol aimed at detecting prevalent but silent pancreatic neoplasms using a combination of endoscopic ultrasound, pancreatic juice collection after iv-secretin stimulation, spiral CT and CA19-9 levels along with counseling regarding their cancer risk. A serum based marker panel with sufficient sensitivity and specificity could facilitate the screening of these individuals at high risk of developing a deadly cancer.

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

References:

The following specific documents, also incorporated herein by reference, are indicated in the above discussion and examples by a number in parentheses corresponding the below numerical listing.
1. AGA American Gastroenterological Association medical position statement: Epidemiology, diagnosis, and treatment of pancreatic ductal adenocarcinoma. Gastroenterology, 117: 1463-1484, 1999.
2. Greenlee, R. T., Hill-Hannon, M B., Murray, T., Thun, M. Cancer statistics. CA Cancer J Clin., 51: 15-36, 2001.
3. Rosty, C. and Goggins, M. Early detection of pancreatic carcinoma. Hematol. Oncol. Clin. North. Am., 16: 37-52, 2002.
4. Yeo, C. J., Cameron, J. L., Lillemoe, K. D., and Sitzmann, J. V. Pancreaticoduodenectomy for cancer of the head of the pancreas.201 patients. Ann Surg, 221: 721-731, 1995.
5. Tersmette, A. C., Petersen, G. M., Offerhaus, G. J. A., Falatko, F. C., Brune, K., Goggins, M., Rozenblum, E., Wilentz, R. E., Yeo, C. J., Cameron, J. L., Kern, S. E., and Hruban, R. H. Increased risk of pancreatic cancer among first-degree relatives of patients with familial pancreatic cancer. Clin. Cancer Res., 7: 738-744, 2001.
6. Giardiello, F. M., Brensinger, J. D., Tersmette, A. C., Goodman, S. N., G. M., P., S. V., B., Cruz-Correa, M., and Offerhaus, G. J. A. Very high risk of cancer in familial Peutz-Jeghers syndrome. Gastroenterology, 119: 1447-1453, 2000.
7. Lowenfels A B, M. P., DiMagno E P, Elitsur Y, Gates L K Jr, Perrault J, Whitcomb D C. Hereditary pancreatitis and the risk of pancreatic cancer. International Hereditary Pancreatitis Study Group. J Natl Cancer Inst., 89: 442-446, 1997.
8. Pleskow, D. K., Berher, H. J., Gyves, J., Allen, E., McLean, A., and Podolsky, D. K. Evaluation of a serologic marker, CA19-9, in the diagnosis of pancreatic cancer. Ann Intern Med, 110: 704-709, 1989.
9. Goggins M, C. M., Hruban R. Can we screen high-risk individuals to detect early pancreatic carcinoma? J Surg Oncol., 74: 243-248, 2000.
10. Weinberger S R, D. E., Fung E T. Current achievements using ProteinChip Array technology. Curr Opin Chem Biol. 2002 Feb;6(1):86-91., 6: 86-91, 2002.
11. Srinivas P R, V. M., Zhao Y, Srivastava S. Proteomics for cancer biomarker discovery. Clin Chem., 48: 1160-1169, 2002.
12. Petricoin E F, A. A., Hitt B A, Levine P J, Fusaro V A, Steinberg S M, Mills G B, Simone C, Fishman D A, Kohn E C, Liotta L A. Use of proteomic patterns in serum to identify ovarian cancer. Lancet, 359: 572-577, 2002.
13. Rai A J, Z. Z., Rosenzweig J, Shih leM, Pham T, Fung E T, Sokoll L J, Chan D W. Proteomic approaches to tumor marker discovery. Arch Pathol Lab Med., 126: 1518-1526, 2002.
14. Li J, Z. Z., Rosenzweig J, Wang Y Y, Chan D W. Proteomics and bioinformatics approaches for identification of serum biomarkers to detect breast cancer. Clin Chem., 48: 1296-1304, 2002.
15. Adam B L, Q. Y., Davis J W, Ward M D, Clements M A, Cazares L H, Semmes O J, Schellhammer P F, Yasui Y, Feng Z, Wright G L Jr. and 1;62(13):3609-14., C. R. J. Serum protein fingerprinting coupled with a pattern-matching algorithm distinguishes pancreatic cancer from benign pancreatic hyperplasia and healthy men. Cancer Res., 62: 3609-3614, 2002.
16. Petricoin E F 3rd, O. D., Paweletz C P, Ardekani A, Hackett P S, Hitt B A, Velassco A, Trucco C, Wiegand L, Wood K, Simone C B, Levine P J, Linehan W M, Emmert-Buck M R, Steinberg S M, Kohn E C, Liotta L A. Serum proteomic patterns for detection of pancreatic cancer. J Natl Cancer Inst., 94: 1576-1578, 2002.
17. Vlahou A, S. P., Mendrinos S, Patel K, Kondylis F I, Gong L, Nasim S, Wright Jr G L Jr. Development of a novel proteomic approach for the detection of transitional cell carcinoma of the bladder in urine. Am J Pathol., 158: 1491-1502, 2001.
18. Rosty C, C. L., Kuzdzal S, Baldwin W M, Zahurak M L, Carnot F, Chan D W, Canto M, Lillemoe K D, Cameron J L, Yeo C J, Hruban R H, Goggins M Identification of hepatocarcinoma-intestine-pancreas/pancreatitis-associated protein I as a biomarker for pancreatic ductal adenocarcinoma by protein biochip technology. Cancer Res., 62: 1868-1875, 2002.
19. Fung E T, E. C. ProteinChip clinical proteomics: computational challenges and solutions. Biotechniques, Supp:, 2002.
20. Boguski M S, M. M. Biomedical informatics for proteomics. Nature, 422: 233-237, 2003.
21. Kanehisa M, B. P. Bioinformatics in the post-sequence era. Nat Genet., 33:305-310, 2003.
22. Iacobuzio-Donahue, C. A., Ryu, B., Hruban, R. H., and Kern, S. E. Exploring the host desmoplastic response to pancreatic carcinoma. Am. J. Pathol., 160:91-99, 2002.
23. Valerio A, B. D., Mazza S, Baldo G, Tiengo A, Pedrazzoli S, Seraglia R, Plebani M. Serum protein profiles of patients with pancreatic cancer and chronic pancreatitis: searching for a diagnostic protein pattern. Rapid Commun Mass Spectrom., 15: 2420-2425, 2001.
24. Adam, B., Qu, Y., Davis, J., Ward, M., Clements, M., Cazares, L., Semmes, O., Schellhammer, P., Yasui, Y., Feng, Z., and Wright, G. J. Serum protein fingerprinting coupled with a pattern-matching algorithm distinguishes pancreatic cancer from benign pancreatic hyperplasia and healthy men. Cancer Res., 62: 3609-3614, 2002.
25. Chen G, G. T., Huang C C, Thomas D G, Shedden K A, Taylor J M, Kardia S L, Misek D E, Giordano T J, lannettoni M D, Orringer M B, Hanash S M, Beer D G.

Proteomic analysis of lung adenocarcinoma: identification of a highly expressed set of proteins in tumors. Clin Cancer Res., 8: 2298-2305, 2002.

1. A method of qualifying pancreatic cancer status in a subject comprising:
(a) measuring at least one biomarker in a sample from the subject, wherein the biomarker is selected from the group consisting of
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and combinations thereof, and
(b) correlating the measurement with pancreatic cancer status.

2. The method of claim 1 further comprising:
(c) managing subject treatment based on the status.

3. The method of claim 2, wherein managing subject treatment is selected from ordering more tests, performing surgery, and taking no further action.

4. The method of claim 2 further comprising:
(d) measuring the at least one biomarker after subject management.

5. The method of claim 1 wherein the pancreatic cancer status is selected from the group consisting of the subject's risk of cancer, the presence or absence of disease, the type of disease, the stage of disease and the effectiveness of treatment of disease.

6-21. (canceled).

22. The method of claim 1 further comprising measuring a known biomarker.

23. The method of claim 22, wherein the known biomarker is CA19-9.

24. The method of any of claim 1 wherein the marker is detected by mass spectrometry.

25. The method of any of claim 1 wherein the marker is detected by capturing the marker on a biochip having an affinity surface and detecting the captured marker by SELDI.

26-28. (canceled).

29. The method of claim 1 wherein the patient sample is selected from the group consisting of blood, blood plasma, serum, urine, tissue, cells, organs and seminal fluids.

30 (canceled).

31. The method of claim 1 comprising:
generating data on immobilized subject samples on a biochip, by subjecting said biochip to laser ionization and detecting intensity of signal for mass/charge ratio; and,
transforming the data into computer readable form;
executing an algorithm that classifies the data according to user input parameters, for detecting signals that represent biomarkers present in pancreatic cancer patients and are lacking in non-cancer subject controls.

32. The method of claim 1 wherein one or more of the biomarkers are detected using laser desorption/ionization mass spectrometry, comprising:
providing a probe adapted for use with a mass spectrometer comprising an adsorbent attached thereto;
contacting the subject sample with the adsorbent;
desorbing and ionizing the biomarker or biomarkers from the probe; and,
detecting the deionized/ionized markers with the mass spectrometer.

33: The method of claim 32, wherein the adsorbent is hydrophobic, hydrophilic, ionic or metal chelate adsorbent.

34. The method of claim 33, wherein the adsorbent is comprised of copper.

35. The method of claim 32, wherein the adsorbent is an antibody, single- or double stranded oligonucleotide, amino acid, protein, peptide or fragments thereof.

36. The method of claim 1, wherein at least one or more protein biomarkers are detected using immunoassays.

37. A process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers, wherein the biomarker is selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and combinations thereof.

38-54. (canceled).

55. A protein purified on a biochip selected from:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD, and
Marker XXXII: having a molecular weight of about 8.754 kD.

56-73. (canceled).

74. A method comprising:
(a) measuring a plurality of biomarkers in a sample from the subject, wherein the biomarkers are selected from the group consisting of:
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD, and
Marker XXXII: having a molecular weight of about 8.754 kD, and combinations thereof.

75-104. (canceled).

105. A method for identifying a compound capable of treating pancreatic cancer comprising:
a) contacting at least one biomarker selected from the group consisting of
Marker I: having a molecular weight of about 3.667 kD
Marker II: having a molecular weight of about 7.441 kD
Marker III: having a molecular weight of about 3.146 kD
Marker IV: having a molecular weight of about 12.861 kD
Marker V: having a molecular weight of about 3.760 kD
Marker VI: having a molecular weight of about 4.053 kD
Marker VII: having a molecular weight of about 5.884 kD
Marker VIII: having a molecular weight of about 6.081 kD
Marker IX: having a molecular weight of about 3.473 kD
Marker X: having a molecular weight of about 5.903 kD
Marker XI: having a molecular weight of about 8.563 kD
Marker XII: having a molecular weight of about 16.008 kD
Marker XIII: having a molecular weight of about 4.159 kD
Marker XIV: having a molecular weight of about 4.179 kD
Marker XV: having a molecular weight of about 7.607 kD
Marker XVI: having a molecular weight of about 4.277 kD
Marker XVII: having a molecular weight of about 4.639 kD
Marker XVIII: having a molecular weight of about 6.093 kD
Marker XIX: having a molecular weight of about 7.463 kD
Marker XX: having a molecular weight of about 9.132 kD
Marker XXI: having a molecular weight of about 3.885 kD
Marker XXII: having a molecular weight of about 3.967 kD
Marker XXIII: having a molecular weight of about 8.929 kD
Marker XXIV: having a molecular weight of about 3.370 kD
Marker XXV: having a molecular weight of about 3.441 kD
Marker XXVI: having a molecular weight of about 10.055 kD
Marker XXVII: having a molecular weight of about 3.510 kD
Marker XXVIII: having a molecular weight of about 9.120 kD
Marker XXIX: having a molecular weight of about 7.294 kD
Marker XXX: having a molecular weight of about 8.866 kD
Marker XXXI: having a molecular weight of about 9.401 kD
Marker XXXII: having a molecular weight of about 8.754 kD, and combinations thereof
with a test compound; and
b) determining whether the test compound binds to the biomarker, wherein a compound that binds to the biomarker is identifies as a compound capable of treated pancreatic cancer.

106-112. (canceled).
